(19) United States
(12) Patent Application Publication
(10) Pub. No.: US 2012/0082655 A1
(43) Pub. Date: Apr. 5, 2012

(54) Downregulation of SPY1 by p53 as an essential component of p53-mediated effects

(76) Inventors: Espanta Jalili, Windsor (CA); Dorota Lubanska, Windsor (CA); Lisa Porter, Windsor (CA)

(21) Appl. No.: 13/137,440

(22) Filed: Aug. 16, 2011

Related U.S. Application Data
(60) Provisional application No. 61/374,422, filed on Aug. 17, 2010.

(51) Int. Cl. A61K 38/45; A61P 35/00; A61K 38/48; C12N 5/071; A61K 38/17

(52) U.S. Cl. 424/94.5; 435/375; 514/19.3; 424/94.63; 435/7.23

(57) ABSTRACT

The present invention relates to a novel method of treating or preventing cancer as well as a novel method for diagnosing or monitoring cancer, wherein the cancer is caused by delayed entry to cellular senescence. More particularly, the present invention relates to a novel method of treating or preventing cancer, comprising a step of administering an agent selected to degrade, inhibit or downregulate Spy1 in a cell. The present invention also relates to a novel method of diagnosing or monitoring cancer, comprising the steps of treating a cell with UV radiation and measuring amounts of a Spy1 protein and a p53 protein, or a ratio thereof.

### RELATED APPLICATIONS

This application claims benefit under 35 U.S.C., §119(e) to U.S. Provisional application No. 61/374,422 filed on Aug. 17, 2010.

### SCOPE OF THE INVENTION

The present invention relates to a novel method of treating or preventing cancer as well as a novel method for diagnosing or monitoring cancer, wherein the cancer may be for example a cancer caused by delayed entry to cellular senescence. More preferably, the present invention relates to a novel method of treating or preventing cancer, comprising a step of administering an agent selected to degrade, inhibit or downregulate a Spy1 protein in a cell. More preferably, the present invention also relates to a novel method of diagnosing and/or monitoring cancer, comprising the steps of treating a cell with UV radiation and measuring the amounts of a Spy1 protein and a p53 protein, or a ratio thereof.

### BACKGROUND OF THE INVENTION

Maintenance of DNA integrity is essential for viability of organisms. As a protection mechanism, and to respond to threats to DNA integrity such as DNA damage, critically shortened or dysfunctional telomeres, protooncogene activation and replicative stress, cells of an organism trigger a DNA damage response (“DDR”) to initiate a host of cellular responses including DNA repair, cell cycle arrest, cellular senescence and apoptosis.

For example, under DDR cellular senescence may involve signaling kinases, ATM and ATR, activating transducer kinases, Chk1 and Chk2. Chk1 and Chk2 in turn activate the tumor suppressor protein, p53, by introducing post-translational modifications to p53 which involve changes to protein stability, DNA binding capabilities, subcellular localization, and tetramerization. Ultimately, activated p53 regulates a number of genes whose protein products are involved in cell cycle arrest, DNA repair and apoptosis. One of p53-regulated protein products include p21 which inhibit the activity of cyclin-dependent kinases (CDKs) and prevent cell cycle transition.

It is appreciated that Spy1 and RINGO family of proteins are cell cycle regulators, which may play a role in meiotic progression and cell proliferation. In particular, Spy1 is may be involved in a number of cancer-inducing activities including 1) activating CDKs, 2) inhibiting or overriding DNA damage-induced apoptosis, 3) bypassing replicative and G2/M cell cycle checkpoints, and 4) preventing repair of cyclobutane pyrimidine dimers.

Although the effect of Spy1 on DDR-induced apoptosis has been appreciated, less is known about the effect of Spy1 on cellular senescence. Cellular senescence, or replicative senescence, may be defined as an arrest or loss of the ability of a cell to divide, and which may be triggered by DNA damages and telomere shortening resulting from cell replication.

### SUMMARY OF THE INVENTION

The applicant seeks to provide methods and compositions which regulate the effect Spy1 may have on cellular senescence, and/or tumor suppression. The applicant has appreciated that an understanding of the relationship between Spy1 and cellular senescence may advantageously provide improved methods, uses and compositions for treating cancer and more preferably cancer caused by delayed entry into cellular senescence.

The applicant having conducted extensive studies and research has discovered that during DDR, Spy1 overrides the effect of p53 on cellular cycle regulation by decreasing its transcriptional activities necessary to initiate cellular senescence. As a result, cells overexpressing or having lost or reduced ability to degrade, inhibit or downregulate Spy1 proteins have delayed entry into cellular senescence, leading to increased number of replicated cells having significant DNA damages and which are potentially cancerous.

The applicant has also discovered that during DDR, the levels of endogenous Spy1 and p53 proteins are inversely regulated. More specifically, the level of endogenous Spy1 decreases and then increases when irradiated with UV radiation to induce DNA damages and cellular senescence; whereas the level of endogenous p53 increases and then decreases. Further, Spy1 has been discovered to significantly reduce the transcriptional activities of p53 when irradiated with the same UV radiation; however, the transcriptional activities were later seen to recover. These findings support that cellular mechanisms exist to downregulate Spy1 and upregulate p53 during DDR and cellular senescence, and which permits recovery from DDR after successful DNA repair.

It is appreciated that p53 plays a role in a negative feedback system which allows for cellular recovery from DDR following successful DNA repair. The applicant has discovered that p53 does not, however, directly provide regulation of Spy1. Rather in a most preferred mode, p53 may be used to effect and/or activate Chk2 to downregulate the levels of Spy1 via modification within the C-terminal region of Spy1 and ubiquitin-mediated degradation by 26S proteosome.

It is therefore an object of the present invention to provide a new method of downregulating a Spy1 protein in a cell.

A further object of the present invention is to provide a new method and a new composition for preventing or treating cancer, which may be, for example, caused by delayed entry into cellular senescence.

A yet further object of the present invention is to provide a new method of diagnosing or monitoring cancer, which may be, for example, caused by delayed entry into cellular senescence.

A yet further object of the present invention is to provide a new method of detecting DNA damages in a cell.

In one aspect, the present invention provides a method of downregulating a Spy1 protein in a cell, the method comprising the step of increasing a p53 protein and a Chk2 protein in the cell, wherein the p53 protein causes the Chk2 protein to cause degradation of the Spy1 protein.

In another aspect, the present invention provides use of a p53 protein in an amount selected to downregulate a Spy1 protein in a cell, wherein the p53 protein causes a Chk2 protein to cause degradation of the Spy1 protein.

Preferably, the Chk2 protein causes degradation of the Spy1 protein by a modification of the Spy1 protein, mostly preferably, at amino acids 217 to 222.

Preferably, the Spy1 protein is degraded by a 26S proteosome. The Spy1 protein may also be preferably targeted for degradation in an N-terminal region by a ubiquitin.

Preferably, the p53 protein in the cell is increased or present in an amount selected to inhibit or reduce cellular replication, or to treat cancer, which may be, for example, caused by delayed entry of the cell into cellular senescence.

In yet another aspect, the present invention provides a method of treating or preventing cancer, the method comprising the step of administering a therapeutically effective amount of an agent selected to downregulate a Spy1 protein in a cell.

In yet another aspect, the present invention provides a composition for use in the treatment or prevention of cancer, the composition comprising a pharmaceutically acceptable carrier and an agent selected to downregulate a Spy1 protein in a cell.

In yet another aspect, the present invention provides use of an agent for the treatment or prevention of cancer, wherein the agent is selected to downregulate a Spy1 protein in a cell.

Preferably, the cancer is a cancer caused by delayed entry of the cell into cellular senescence.

The agent may preferably include one or more of a p53 protein, a Chk2 protein, and a S26 proteosome. The p53 protein is most preferably present in an amount which is selected to cause the Chk2 protein to cause degradation of the Spy1 protein.

In yet another aspect, the present invention provides a method of diagnosing or monitoring cancer, the method comprising the steps of extracting a cell from a patient, treating the cell with UV radiation, and measuring amounts of a Spy1 protein and a p53 protein, or a ratio thereof. Preferably, the cancer is caused by delayed entry into cellular senescence. Preferably, the UV radiation comprises a dose of 50 J/m² of UVC radiation. Preferably, the amounts or the ratio of the Spy1 protein and the p53 protein are measured at different time points.

In yet another aspect, the present invention provides a method of detecting DNA damages in a cell, the method comprising the steps of extracting a cell from a patient and measuring amounts of a Spy1 protein and a p53 protein, or a ratio thereof. Preferably, the amounts or the ratio of the Spy1 protein and the p53 protein are measured at different time points.

### BRIEF DESCRIPTION OF THE DRAWINGS

Reference may now be had to the following detailed description, taken together with the accompanying drawings, in which:

FIG. 1a illustrates a western blot of Spy1 at different cell passages (indicated on top) of HFF-1 cells transfected with a control vector (HFF-1-pLXSN) or Spy1 vector (HFF-1-Spy1).

FIG. 1b illustrates a western blot of p53 at different cell passages (indicated on top) of HFF-1 cells transfected with a control vector (HFF-1-pLXSN) or Spy1 vector (HFF-1-Spy1).

FIG. 1c illustrates a western blot of GAPDH loading control at different cell passages (indicated on top) of HFF-1 cells transfected with a control vector (HFF-1-pLXSN) or Spy1 vector (HFF-1-Spy1).

FIG. 2 illustrates a graph showing population doubling times at different cell passages of HFF-1 cells transfected with a control vector (pLSXN) or a vector containing Spy1 gene.

FIG. 3a are microscopic views taken at different cell passages of non-transfected control HFF-1 cells.

FIG. 3b are microscopic views taken at different cell passages of HFF-1 cells transfected with a control vector (HFF-1-pLXSN).

FIG. 3c are microscopic views taken at different cell passages of HFF-1 cells transfected with Spy1 vector and overexpressing Spy1 (HFF-1-Spy1).

FIG. 4 are microscopic views taken at cell passage 58 of HFF-1 cells transfected with a control vector (HFF-1-pLXSN, left) or a Spy1 vector (HFF-1-Spy1, right).

FIG. 5a illustrates western blots of Spy1 at cell passages 50, 65 and passage senescence of HFF cells.

FIG. 5b illustrates western blots of p53 at cell passages 50, 65 and passage senescence of HFF cells.

FIG. 5c illustrates western blots of actin loading control at cell passages 50, 65 and passage senescence of HFF cells:

FIG. 5d illustrates a graph showing the corresponding levels of Spy1 and p53 (as shown in FIGS. 5a and 5b) determined by densitometry at different cell passages of HFF cells.

FIG. 6a illustrates western blots of Spy1 at different time points from the time of irradiating U2OS cell line with UVC light (50 J/m²).

FIG. 6b illustrates western blots of p53 at different time points from the time of irradiating U2OS cell line with UVC light (50 J/m²).

FIG. 6c illustrates western blots of actin loading control at different time points from the time of irradiating U2OS cell line with UVC light (50 J/m²).

FIG. 6d illustrates a graph showing the corresponding levels Spy1 and p53 (as shown in FIGS. 6a and 6b) determined by densitometry at different cell passages of U2OS cell line.

FIG. 7a illustrates western blots of Spy1 of NIH3T3 cells, U2OS cells and HEK-293 cells transfected with controls, Myc-Spy1-pCS3 Flag-p53, or both of Myc-Spy1-pCS3 and Flag-p53.

FIG. 7b illustrates western blots of p53 of NIH3T3 cells, U2OS cells and HEK-293 cells transfected with controls, Myc-Spy1-pCS3 Flag-p53, or both of Myc-Spy1-pCS3 and Flag-p53.

FIG. 7c illustrates western blots of actin loading control of NIH3T3 cells, U2OS cells and HEK-293 cells transfected with controls, Myc-Spy1-pCS3 Flag-p53, or both of Myc-Spy1-pCS3 and Flag-p53.

FIG. 8a illustrates western blots of Spy1 at different time points after cyclohexamide (25 μg) treatment of HEK-293 cells transfected with controls, Myc-Spy1-pCS3, Flag-p53, or both of Myc-Spy1-pCS3 and Flag-p53.

FIG. 8b illustrates western blots of p53 at different time points after cyclohexamide (25 μg) treatment of HEK-293 cells transfected with controls, Myc-Spy1-pCS3, Flag-p53, or both of Myc-Spy1-pCS3 and Flag-p53.

FIG. 8c illustrates western blots of actin loading control at different time points after cyclohexamide (25 μg) treatment of HEK-293 cells transfected with controls, Myc-Spy1-pCS3, Flag-p53, or both of Myc-Spy1-pCS3 and Flag-p53.

FIG. 9a illustrates western blots for Spy1 of U2OS cells transfected with pCS3 control or Myc-Spy1-pCS3, and which are either radiated or not radiated with UVC radiation (50 J/m²).

FIG. 9b illustrates western blots for p53 of U2OS cells transfected with pCS3 control or Myc-Spy1-pCS3, and which are either radiated or not radiated with UVC radiation (50 J/m²).

FIG. 9c illustrates western blots for actin loading control of U2OS cells transfected with pCS3 control or Myc-Spy1-pCS3, and which are either radiated or not radiated with UVC radiation (50 J/m²).

FIG. 10a illustrates western blots of Spy1 of HCT116 p53^{+/+} cells transfected with pCS3 control, Myc-Spy1-pCS3, or Myc-DMA-pCS3, and which are either not treated or treated with 50 J/m² of UVC radiation for 12 hours or 24 hours.

FIG. 10b illustrates western blots of actin loading control of HCT116 p53^{+/+} cells transfected with pCS3 control, Myc-Spy1-pCS3, or Myc-DMA-pCS3, and which are either not treated or treated with 50 J/m² of UVC radiation for 12 hours or 24 hours.

FIG. 11a illustrates western blots of Spy1 for Saos-2 cells transfected with pCS3 control or Myc-Spy1-pCS3, and which are either treated or not treated with UV radiation (50 J/m²) for 24 hours.

FIG. 11b illustrates western blots of p53 for Saos-2 cells transfected with pCS3 control or Myc-Spy1-pCS3, and which are either treated or not treated with UV radiation (50 J/m²) for 24 hours.

FIG. 11c illustrates western blots of actin loading control for Saos-2 cells transfected with pCS3 control or Myc-Spy1-pCS3 and which are either treated or not treated with UV radiation (50 J/m²) for 24 hours.

FIG. 12a illustrates western blots for Spy1 for HCT116 p53^{+/+} cells transfected with pCS3 control or Myc-Spy1-pCS3, and which are either treated or not treated with UVC radiation (50 J/m²) in the presence or absence of 100 nm UCN-01.

FIG. 12b illustrates western blots for p53 for HCT116 p53^{+/+} cells transfected with pCS3 control or Myc-Spy1-pCS3, and which are either treated or not treated with UVC radiation (50 J/m²) in the presence or absence of 100 nm UCN-01.

FIG. 12c illustrates western blots for actin loading control for HCT 116 p53^{+/+} cells transfected with pCS3 control or Myc-Spy1-pCS3, and which are either treated or not treated with UVC radiation (50 J/m²) in the presence or absence of 100 nm UCN-01.

FIG. 13a illustrates western blots for Spy1 for HEK-293 cells transfected with pCS3 control or Myc-Spy1-pCS3, and which are either treated or not treated with 30 or 50 J/m² of UVC radiation in the presence or absence of Chk2 inhibitor II.

FIG. 13b illustrates western blots for p53 for HEK-293 cells transfected with pCS3 control or Myc-Spy1-pCS3, and which are either treated or not treated with 30 or 50 J/m² of UVC radiation in the presence or absence of Chk2 inhibitor II.

FIG. 13c illustrates western blots for actin loading control for HEK-293 cells transfected with pCS3 control or Myc-Spy1-pCS3, and which are either treated or not treated with 30 or 50 J/m² of UVC radiation in the presence or absence of Chk2 inhibitor II.

FIG. 14a illustrates a graph showing the numbers of live cells counted by trypan blue exclusion after treatment with UV radiation of varying energy and duration.

FIG. 14b illustrates a graph showing the numbers of dead cells counted by trypan blue exclusion after treatment with UV radiation of varying energy and duration.

FIG. 15a illustrates a graph showing the numbers of live and dead HCT116 p21^{+/+} cells transfected with pCS3 control or Myc-Spy1-pCS3, and which are either mock treated or treated with UV radiation (50 J/m²).

FIG. 15b illustrates a graph showing the numbers of live and dead HCT116 p21^{-/-} (right) cells transfected with pCS3 control or Myc-Spy1-pCS3, and which are either mock treated or treated with UV radiation (50 J/m²).

FIG. 16a illustrates western blots for P³² histone H1 of HEK-293 transfected with pCS3 control, Myc-Spy1-pCS3, Flag-p21, or both of Myc-Spy1-pCS3 and Flag-p21.

FIG. 16b illustrates western blots for CDK2 of HEK-293 transfected with pCS3 control, Myc-Spy1-pCS3, Flag-p21, or both of Myc-Spy1-pCS3 and Flag-p21.

FIG. 17a illustrates western blots of p21 at different time points after 25 μg/mL cyclohexamide treatment of HEK-293 cells transfected with Myc-Spy1-pCS3, Flag-p21, or both of Myc-Spy1-pCS3 and Flag-p21.

FIG. 17b illustrates western blots of actin loading control at different time points after 25 μg/mL cyclohexamide treatment of HEK-293 cells transfected with Myc-Spy1-pCS3, Flag-p21, or both of Myc-Spy1-pCS3 and Flag-p21.

FIG. 17c illustrates a bar graph showing the corresponding levels of p21 (as shown in FIGS. 17a and 17b) determined by densitometry.

FIG. 17d illustrates a line graph showing the corresponding levels of p21 (as shown in FIGS. 17a and 17b) and the rates of p21 degradation determined by densitometry.

FIG. 18a illustrates a graph showing the numbers of NIH3T3 cells transfected with pCS3 Control, Myc-Spy1-pCS3, Flag-p53, or both of Myc-Spy1-pCS3 and Flag-p53.

FIG. 18b illustrates a graph showing the numbers of HEK-293 cells transfected with pCS3 control, Myc-Spy1-pCS3, Flag-p53, or both of Myc-Spy1-pCS3 and Flag-p53.

FIG. 19a illustrates a graph showing the numbers of U2OS cells transfected'with pCS3 control or Myc-Spy1-pCS3, and which are either treated or not treated with UVC radiation (50 J/m²).

FIG. 19b illustrates a graph showing the numbers of Saos-2 cells transfected with pCS3 control or Myc-Spy1-pCS3, and which are either treated or not treated with UVC radiation (50 J/m²).

FIG. 20a illustrates western blots of Spy1 for U2OS cells transfected with pCS3 control or Myc-Spy1-pCS3, and which are either mock treated or treated with UV radiation (50 J/m²) for 24 hours.

FIG. 20b illustrates western blots of p53 for U2OS cells transfected with pCS3 control or Myc-Spy1-pCS3, and which are either mock treated or treated with UV radiation (50 J/m²) for 24 hours.

FIG. 20c illustrates western blots of actin loading control for U2OS cells transfected with pCS3 control or Myc-Spy1-pCS3, and which are either mock treated or treated with UV radiation (50 J/m²) for 24 hours.

FIG. 21a illustrates western blots of Spy1 for HEK-293 cells transfected with pCS3 control, Myc-Spy1-pCS3, Myc-Spy1D90A, Myc-Spy1Y107A, both of Myc-Spy1-pCS3 and Flag-p53, both of Myc-Spy1D90A and Flag-p53, both of Myc-Spy1Y107A and Flag-p53, or Flag-p53.

FIG. 21b illustrates western blots of p53 for HEK-293 cells transfected with pCS3 control, Myc-Spy1-pCS3, Myc-Spy1D90A, Myc-Spy1Y107A, both of Myc-Spy1-pCS3 and Flag-p53, both of Myc-Spy1D90A and Flag-p53, both of Myc-Spy1Y107A and Flag-p53, or Flag-p53.

FIG. 21c illustrates western blots of actin loading control for HEK-293 cells transfected with pCS3 control, Myc-Spy1-pCS3, Myc-Spy1D90A, Myc-Spy1Y107A, both of Myc-Spy1-pCS3 and Flag-p53, both of Myc-Spy1D90A and Flag-p53, both of Myc-Spy1Y107A and Flag-p53, or Flag-p53.

FIG. 22 illustrates a graph showing the results of a luciferase assay performed with HCT116 p53^{+/+} cells transfected with pCS3 vector control, Spy1-pCS3 or Spy1-D90A-pCS3 in combination with PG13-Luc and MG15-Luc.

### DETAILED DESCRIPTION OF THE PREFERRED EMBODIMENTS

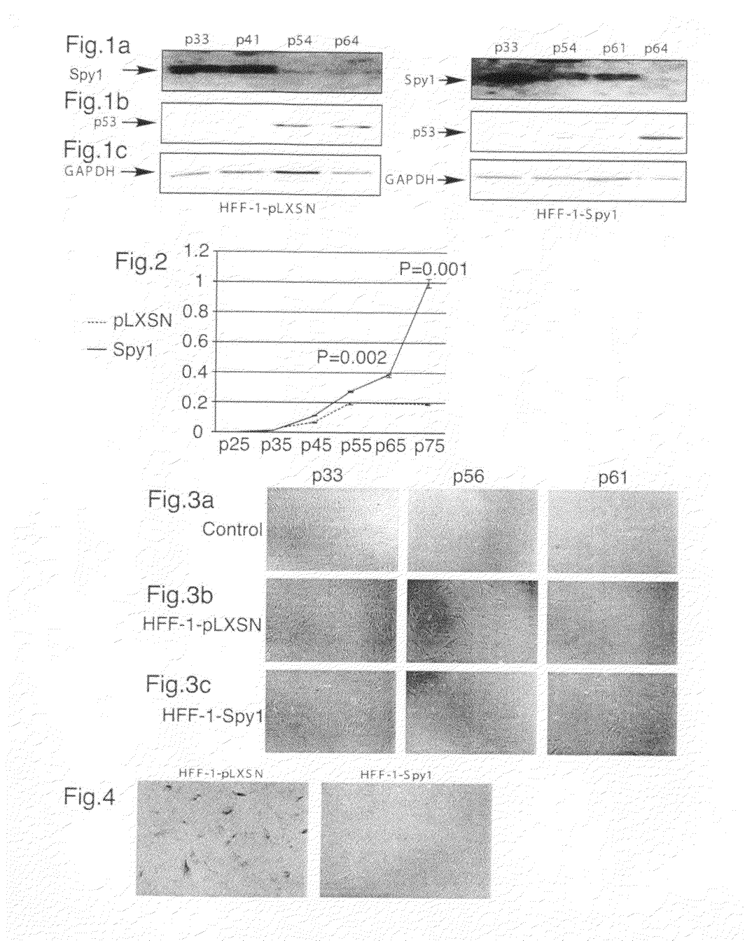

The most preferred embodiments of the present invention are henceforth described with reference to FIGS. 1 to 22. The most preferred embodiments are provided as mere examples which are in no way intended to limit the scope of the present invention. It will be readily apparent to a person skilled in the art that variations and modifications may be made to the most preferred embodiments within the scope of the present invention as described herein.

In one preferred method in accordance with the invention, a patient diagnosed with cancer is administered a composition comprising an agent which includes a p53 protein, a Chk2 protein, and a S26 proteosome, in the treatment of a cancer caused by delayed entry into cellular senescence.

The cancer which is caused by delayed entry into cellular senescence includes, but not limited, to solid tumors and blood born tumors. The cancer may refer to disease of skin tissues, organs, bone, cartilage, blood and vessels. The composition may be used to treat variety of cancer including, but not limited to, cancer of the head, neck, eye, mouth, throat, esophagus, chest, bone, lung, colon, rectum, stomach, prostate, breast, ovaries, kidney, liver, pancreas and brain. The cancer encompasses primary and metastatic cancers.

In addition to the agent, the composition may further contain other anticancer ingredients or drugs which do not impair the functions of the agent. Such anticancer ingredients may include, but not limited to, an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine), an alkylating agent (including but not limited to busulfan, chlorambucil, cyclophosphamide, ifofamide, mechlorethamine, melphalan, and thiotepa), nonclassical akylating agents, platinum containing compounds, bleomycin, an anti-tumor antibiotic, an anthracycline such as doxorubicin and dannomycin, an anthracenedione, topoisomerase II inhibitors, hormonal agents (including but not limited to corticosteriods (dexamethasone, prednisone, and methylprednisone), androgens such as fluoxymesterone and methyltestosterone), estrogens such as diethylstilbesterol, antiestrogens such as tamoxifen, LHRH analogues such as leuprolide, antiandrogens such as flutamdie, aminogluetethimide, megestrol acetate, and medroxyprogesterone, asparaginase, carmustine, lomustine, hexamethyl-melamine, dacarbazine, mitotane, streptozocin, cisplatin, carboplatin, levamasole, and leucovorin. The compounds of the present invention can also be used in combination with enzyme therapy agents and immune system modulators such as an interferon, interleukin, tumor necrosis factor, macrophage colony-stimulating factor and colony stimulating factor.

The composition may be administered to the patient in liquid or solid form by any appropriate route which, for example, may include oral, parenteral, intravenous, intradermal, transdermal, mucosal, subcutaneous, and topical.

The concentration of the agent may depend on absorption, inactivation and excretion rates of the agent as well as other factors known to a person skilled in the art. Specifically, the concentration may range from about 1 to about 95 percent by weight.

It is to be noted that dosage will also vary with the conditions, age, body weight and severity of the cancer to be treated. It will be readily apparent to a person skilled in the art that for each patient, specific dosage regimens could be adjusted over time according to individual needs. The composition or the agent may be administered once or may be divided into a number of smaller doses to be administered at varying intervals of time.

For oral administration, the composition may further include an inert diluent or an edible carrier. They may be enclosed in gelatin capsules or compressed into tablets. Further, the agent may be incorporated with excipients and used in the form of tablets, troches, or capsules. Pharmaceutically compatible binding agents, and/or adjuvant materials may also be included in the composition.

The tablets, capsules, troches and the like can contain any of the following ingredients, or compounds of similar nature: a binder such as microcrystalline cellulose, gum tragacanth or gelatin; an excipient such as starch or lactose; a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to the aforementioned materials, a liquid carrier such as fatty oil. In addition, dosage unit forms can contain various other materials which modify the physical form of the dosage unit, for example, coating of sugar, shellac, or other enteric agents.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioixdants such as ascorbic acid or sodium bisulfite; chelating agents such as ethylenediaminetetraacetic acid; buffers such as acetates, citrates or phosphates; and agents for adjusting tonicity such as sodium chloride and dextrose. The parental preparation can be enclosed in ampoules, disposable syringes or multiple dose vials made of glass or plastic.

For intravenous administration, the composition may further include carriers physiological saline or phosphate buffer saline (PBS).

Suitability of a particular route of administration employed will depend on the physical state of the composition or the agent, and the disease being treated. For example, treatment of cancer on the skin or an exposed mucosal tissue may be more effective if the composition is administered topically, transdermally or mucosally (e.g. by nasal, sublingual, buccal, rectal, or vaginal administration). Treatment of cancer within the body, or prevention of cancers that may spread from one part of the body to another, may be more effective if the composition is administered parenterally or orally. Similarly, parenteral administration may be preferred for the acute treatment of cancer, whereas transdermal or subcutaneous routes of administration may be employed for chronic treatment or prevention of cancer.

The composition may also be prepared with carriers that will protect the agent against rapid elimination from the patient body, such as controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid.

Various methods are known to a person skilled in the art which may be used to prepare the composition.

In a preferred embodiment, cancer which is caused by delayed entry into cellular senescence could be diagnosed or monitored by extracting a cell from a patient, treating the cell with UV radiation, and measuring the ratio of p53 and Spy1 proteins at different time points after the UV treatment step. Preferably, the UV radiation provides a dose of 50 J/m² of UVC radiation to the cell to induce DDR. Preferably, the time profile of the measured ratio of p53 and Spy1 proteins is compared to that of a healthy, non-cancerous cells to determine the presence of any deviation from the latter, as an indication of cancer.

In a preferred embodiment, the presence and the extent of DNA damages to a cell could be measure by extracting the cell of interest and measuring the ratio of p53 and Spy1 proteins at different time points. Preferably, the time profile of the measured ratio of p53 and Spy1 proteins is compared to that of a healthy, non-cancerous cells to determine the presence of any deviation, from the latter as an indication of the presence of DNA damages.

The applicant has appreciated that Spy1's effect on cellular proliferation may be downregulated to provide therapeutic benefits useful in the treatment, diagnosis, and/or prophylaxis of various cancers.

### Clinical Background

### 1. Spy1 Overrides Replicative Senescence.

To determine the necessity for the downregulation of Spy1 during replicative senescence, human foreskin fibroblast cells (HFF-1) were generated to stably overexpress Spy1 (HFF-1-Spy1) or vector control (HFF-1-pLXSN). Four individual HFF-1-Spy1 or HFF-1-pLXSN colonies as well as mock control cells (TIFF-1-Cntl.) were cultured to senescence. Expression was monitored by western blot analysis (shown in FIGS. 1a to 1c) and cell counts were taken via trypan blue exclusion to determine the mean doubling time of each population (shown in FIG. 2). Cell lysates obtained over several passages revealed that endogenous levels of Spy1 in our control stable line were downregulated when p53 levels were rising (shown in FIGS. 1a to 1c, left side). While significantly delayed, Spy1 levels were even seen to decrease at late passages in our stable overexpressing lines (shown in FIGS. 1a to 1c, right side).

Over four separate experiments cells overexpressing Spy1 demonstrated a significant delayed entry into senescence by at least 20 cell passages (shown in FIGS. 3a to 3c). Microscopic analysis of cells at each passage at identical magnification monitored for the onset of senescent morphology (shown in FIGS. 3a to 3c), this was further confirmed through the use of SA-β-Gal staining (shown in FIG. 4).

The data which are illustrated in FIGS. 1 to 4 demonstrates that high levels of Spy1 are capable of overriding senescence incurred via critically shortened telomeres.

### 2. Spy1 and p53 are Inversely Expressed During Cellular Senescence and the DNA Damage Response.

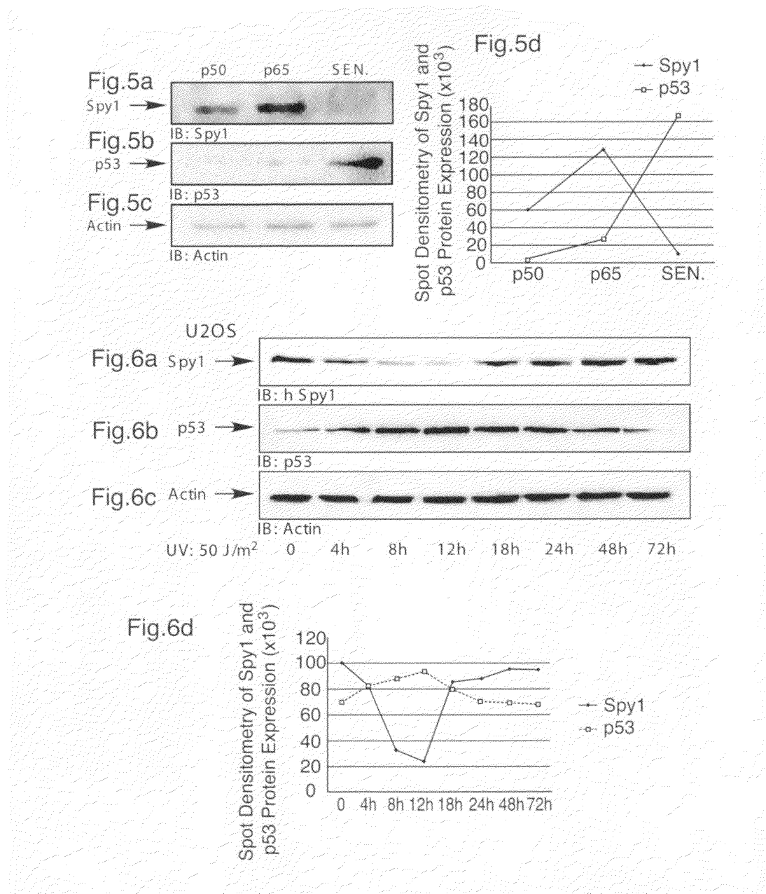

Interestingly, in our stable cell systems shown in FIGS. 1 to 4, it was discovered that even when Spy1 was overexpressed and clearly overriding senescent barriers, Spy1 protein levels began to drop at late passages and p53 protein levels began to accumulate. To further explore the regulation of endogenous Spy1 protein in response to senescent stimuli HFF cells were cultured to senescence (shown in FIG. 5a to 5d) or U2OS cells were exposed to 50 J/m² UVC (shown in FIGS. 6a to 6d).

We see that Spy1 protein levels repeatedly decrease when p53 levels begin to accumulate at late stage HFFs (shown in FIGS. 5a to 5d). Senescence was confirmed using SA-β-Gal staining. In response to doses of UV known to allow for DNA repair and DDR recovery, Spy1 expression is biphasic with levels decreasing approximately 4-8 h after irradiation as p53 accumulates. Similarly Spy1 levels begin to re-accumulate after 12 h post-UV, when p53 levels reduce (shown in FIGS. 6a to 6d).

### 3. Spy1 Protein Levels are Tightly Regulated by p53.

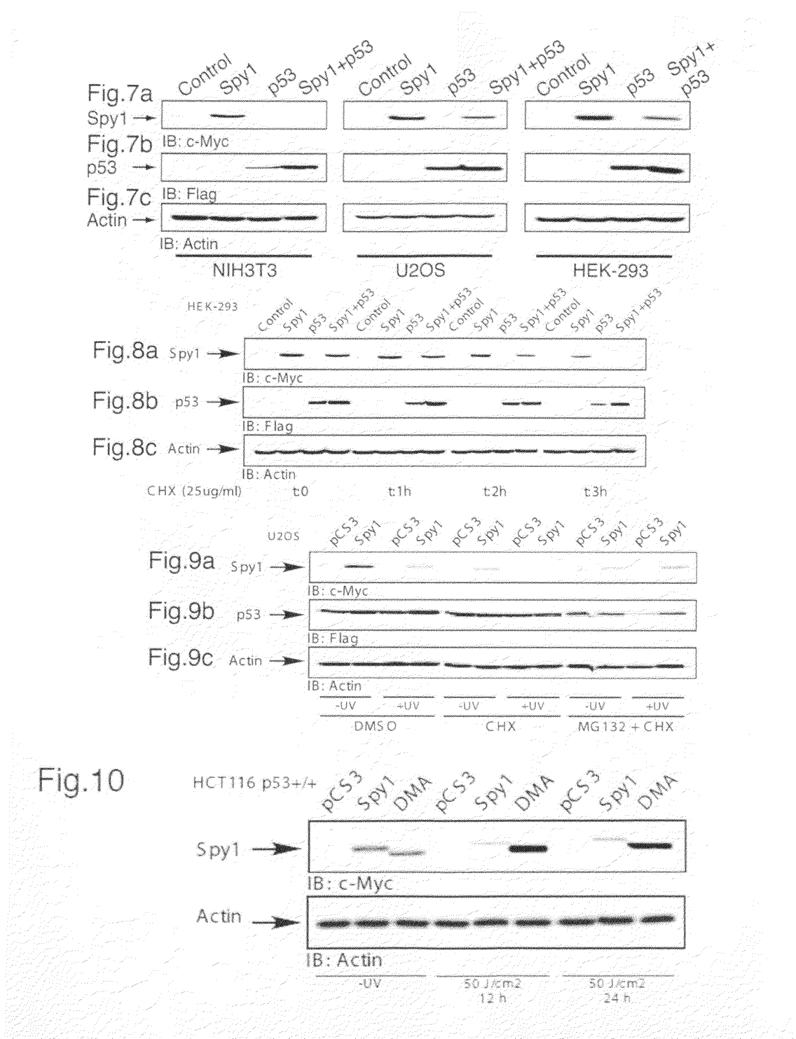

To further determine whether p53 expression plays a direct role in the regulation of Spy1 protein levels, Spy1, p53 or combinations of both were exogenously overexpressed in a number of cell lines. In each case, overall levels of Spy1 protein was significantly depleted in the presence of overexpressed p53 (shown in FIGS. 7a to 7c). To obtain the western blots of FIGS. 7a to 7c, the cells were lysed and analyzed by 10% SDS-PAGE.

Spy1 protein levels were studied in the presence of cycloheximide to determine the necessity of de novo protein synthesis for p53-mediated Spy1 degradation (shown in FIGS. 8a to 8c). To obtain the western blots of FIGS. 8a to 8c, the transfected cells were incubated for 24 hours, treated with cyclohexamide, lysed at different time points, and then analyzed for protein levels. Spy1 protein levels were significantly depleted after 2 h cyclohexamide treatment in the presence of p53, hence the DDR-mediated reduction in Spy1 protein levels occurs in a manner independent of de novo protein synthesis.

Spy1 protein has been appreciated to be degraded in a proteosome-dependent manner. To further determine whether DDR-mediated Spy1 degradation occurs in a proteosome dependent fashion, control or Spy1 overexpressing cells in the presence or absence of 50 J/m² UV damage were treated with vehicle control, cyclohexamide or cyclohexamide with MG132 to inhibit the 26S proteosome (shown in FIGS. 9a to 9c). To obtain the western blots of FIGS. 9a to 9c, the cells were treated with cyclohexamide following a DMSO/MG132 treatment. Cells were harvested 6 hours after the cyclohexamide treatment to monitor Spy1 protein stability. In the presence of MG132 Spy1 protein levels remained stable following UV damage, supporting that DDR-mediated degradation of Spy1 is occurring via a proteosome dependent mechanism.

It has been appreciated that the N-terminal region of Spy1 is essential for targeting the protein for ubiquitin-mediated degradation by the proteosome, although dispensable for all known functional roles of the protein. To test the essentiality of this region a Spy1 deletion mutant lacking 57 aa from the Nterminus region was utilized (DMA). FIGS. 10a and 10b demonstrates that following UV damage wild type Spy1 is degraded, however, the DMA constructs accumulate, demonstrating that indeed this region is essential for DDR-mediated degradation of Spy1.

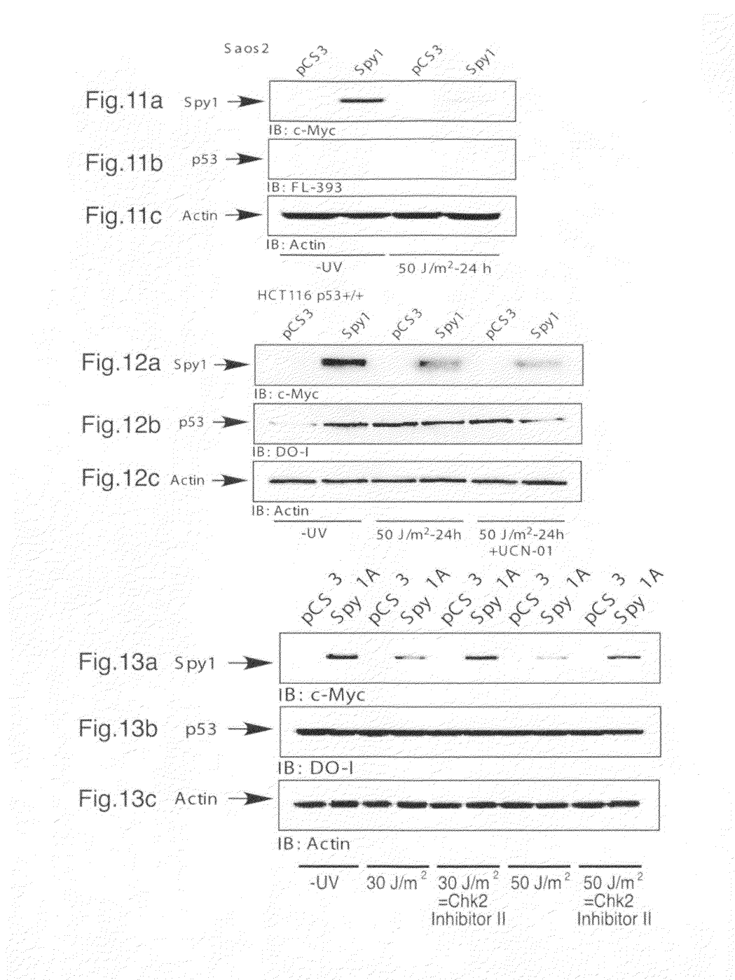

It has been appreciated that p53 levels and activity play a role in the negative feedback signaling required to allow for DDR recovery following successful DNA repair. Hence to determine whether Spy1 degradation is dependent on p53 we utilized an osteosarcoma cell line devoid of endogenous p53, Saos-2 overexpressing control or Spy1 in the presence or absence of 50 J/m² of UV (shown in FIGS. 11a to 11c). The western blots of FIGS. 11a to 11c are obtained using monoclonal c-Myc and DO-1 antibodies. Spy1 protein levels continued to be significantly depleted following UV damage, demonstrating that this response is not dependent on the expression of p53.

Within the C-terminal region of Spy1 we note that there is a consensus site for the DDR transducer kinases Chk1 and Chk2 (LXRXXS) at residues 217 to 222 (LPRGPS) (SEQ ID NO: 2). Hence, cells were damaged with UV in the presence or absence of chemical inhibitors for the DDR transducer kinases Chk1 (UCN-01; shown in FIGS. 12a to 12c) or Chk2 (Chk2 Inhibitor II; shown in FIGS. 13a to 13c) and protein levels of Spy1 were analyzed. For the western blots of FIGS. 12a to 12c, the cells were collected 4 hours after UV radiation. For the western blots of FIGS. 13a to 13c, the cells were harvested 24 hours after inhibitor addition. Spy1 protein levels continued to be depleted following UV damage in the presence of the Chk1 inhibitor (shown in FIGS. 12a to 12c) but levels were significantly higher in the presence of the Chk2 inhibitor (shown in FIGS. 13a to 13c). This suggests that Spy1 protein levels depend on modifications from Chk2.

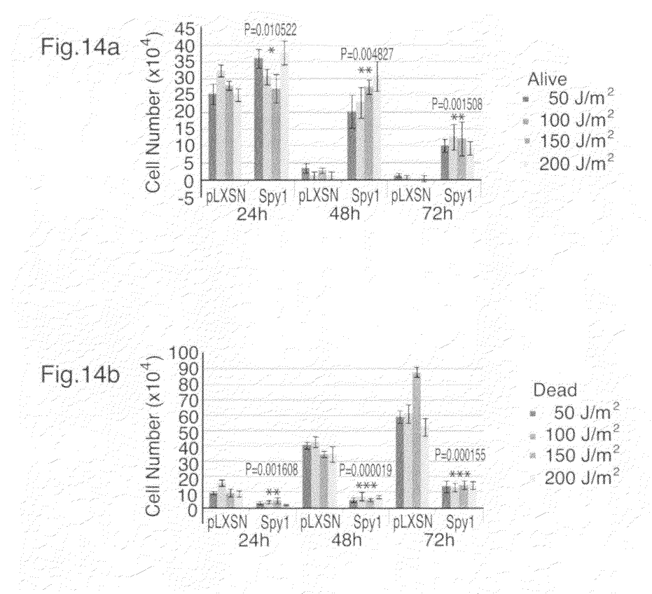

It has been appreciated that Spy1 is capable of overriding DNA damage induced apoptosis. Covering the dose and time range of UV irradiation where we see visible depletion of Spy1 protein levels, it is notable that Spy1 overexpression continues to have a very significant effect on both cell growth and death (shown in FIGS. 14a and 14b). In FIGS. 14a and 14b, the errors represent the mean±S.D. (n=3), and that cell counts for samples overexpressing Spy1 indicate a statistically significant increase from control pLXSN cells (p<0.05). Spy1 significantly overrides even higher doses of irradiation after 72 hr of treatment, demonstrating significantly more live proliferating cells (as shown in FIG. 14a) and reduced numbers of dying cells (as shown in FIG. 14b).

### 4. Spy1 Regulation of p21 Following the DDR.

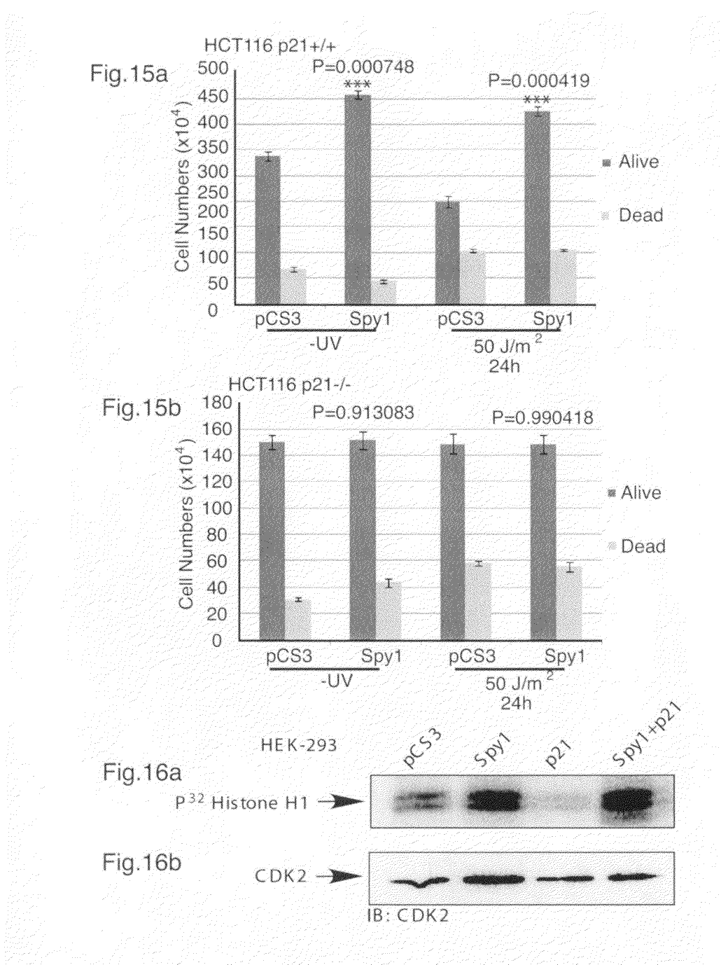

It has been appreciated that Spy1 overrides DDR mediated apoptotic events in a manner dependent on p21. Utilizing the HCT116 p21^{+/+} or p21^{-/-} cell systems, we tested the effects of Spy1 on cell proliferation and cell death using doses of UV capable of inducing senescence with minimal apoptosis (as shown in FIGS. 15a and 15b). For FIGS. 15a and 15b, the cell numbers were assessed 12 hours after UV exposure by trypan blue staining. The bars in FIGS. 15a and 15b represent means standard deviations. In the presence of p21 overexpression of Spy1 significantly enhanced cell proliferation in the presence and absence of UV damage (as shown in FIG. 15a); however these effects were not seen in the p21−/− cell system (as shown in FIG. 15b). Effects on apoptosis seen at this dose/time demonstrated no statistically significant changes (as shown in grey bars in FIGS. 15a and 15b).

It has also been appreciated that Spy1 effects are insensitive to inhibition by p21, hence we also carried out a kinase assay to measure CDK2 activity in the presence of Spy1, p21 or each together (as shown in FIGS. 16a and 16b). To obtain the western blots of FIGS. 16a and 16b, cell lysates were immunoprecipitated with anti-CDK2 antibody and analyzed by histone H1 assay 24 hours after transfection. FIGS. 16a and 16b demonstrate that CDK2 kinase activity remains active in the presence of Spy1 despite expression of p21.

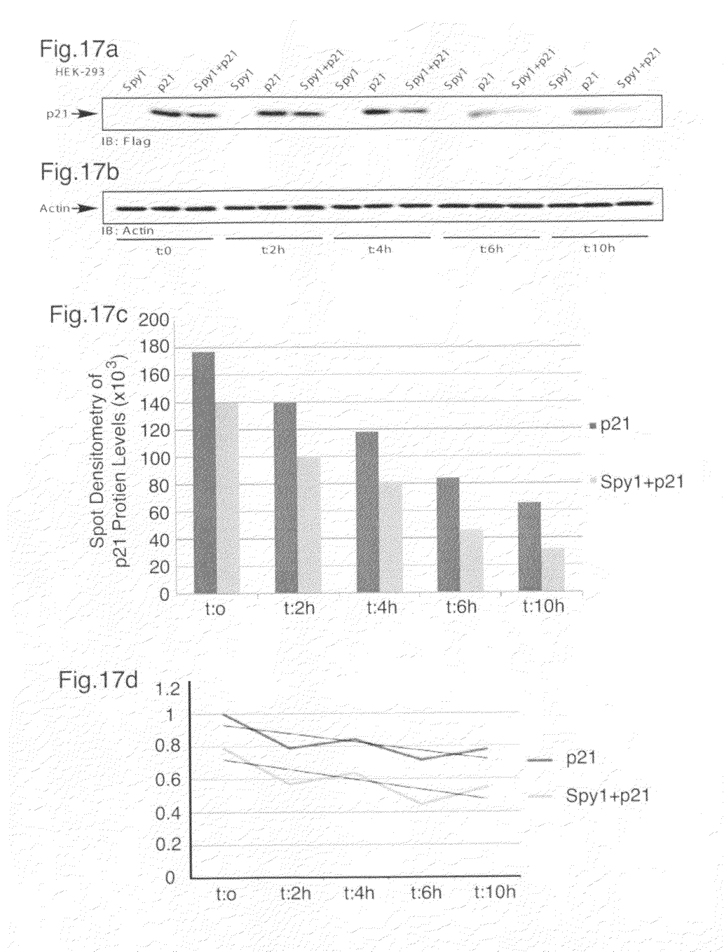

It has been appreciated that p21 protein can be targeted for degradation by CDK2 through phosphorylation on the C-terminal residue S130; and that Spy1 directly regulate CDK2-mediated phosphorylation of the p21 family member p27, which harbours structural and functional similarities with regard to binding interactions with CDKs. Hence, we studied p21 protein levels in the presence of cyclohexamide in cells overexpressing Spy1, p21 or a combination of both (as shown in FIGS. 17a to 17d). For FIGS. 17a to 17d, the data performed in triplicate is expressed in mean±S.D., and the rates of p21 degradation in FIG. 17d are shown as the slope of straight lines. We found a considerable decrease in p21 protein abundance in the presence of Spy1, however, when densitometry was conducted it was noted that there was considerably less p21 protein at time zero. Studying the slope of degradation of p21 in this experiment in association with findings from a pulse chase experiment demonstrated that p21 protein decreased at a similar rate in the presence or absence of Spy1 (as shown in FIG. 17d). Hence, the differences in in initial p21 protein levels may be caused by the regulatory effect of Spy1 on the transcriptional activity of p53 to deplete p21 transcripts.

5. Spy1 Overrides p53-Transcriptional Activity and Cell Cycle Effects.

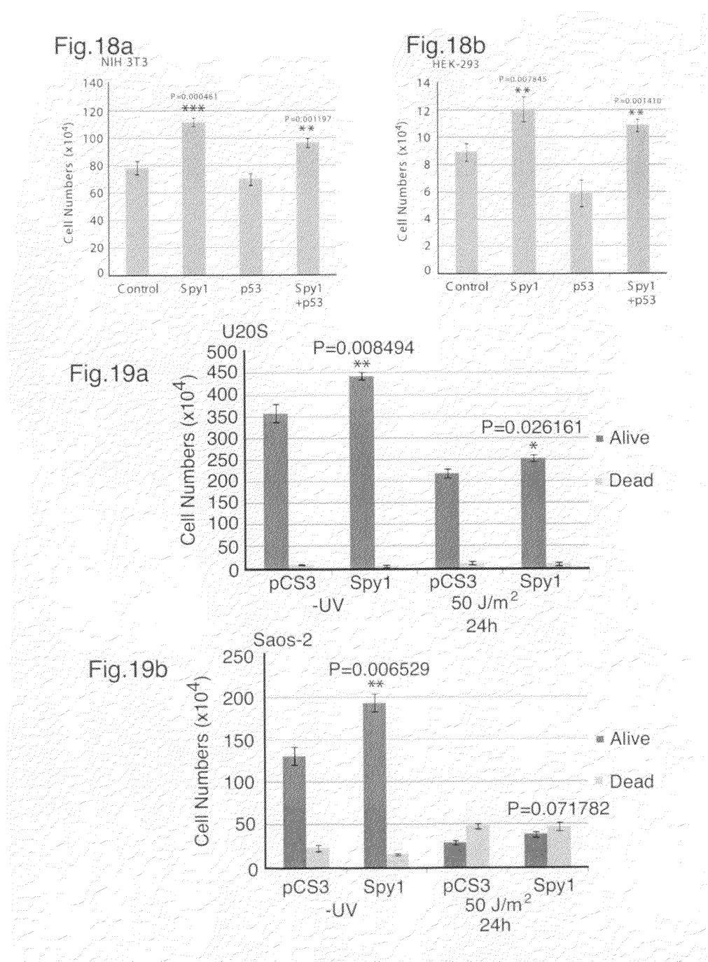

To address whether Spy1 was capable of altering the activities of p53 on cell cycle progression directly we first transfected cells with Spy1, p53 or combinations of both and assessed overall cell growth via trypan blue analysis (as shown in FIGS. 18a and 18b). For FIGS. 18a and 18b, the experiment was performed in triplicate and repeated at least 3 times. The columns in FIGS. 18a and 18b represent overall means±S.D. Spy1 significantly bypassed effects of p53 directly, significantly enhancing cell numbers to greater than that of controls. Western blot for these counts are provided in FIGS. 7a to 7c.

It has been appreciated that Spy1 mediated effects on apoptosis 24 h following exposure to 50 J/m² UVC are dependent on p53 using the HCT116 p53^{+/+} and p53^{-/-} system. To determine whether effects on doses and timing of UV damage demonstrating senescent effects with little induction of apoptosis are also dependent on p53 we utilized the U2OS (p53^{+/+}) and Saos2 (p53^{-/-}) cell systems (as shown in FIGS. 19a and 19b). For FIGS. 19a and 19b, cell viability was determined by trypan blue analysis 24 hours after radiation. Each column in FIGS. 19a and 19b represent overall means±S.D. Spy1 significantly increased cell proliferation following UV damage in the p53^{+/+} cell system (as shown in FIG. 19a) but not in the p53^{-/-} cell system (as shown in FIG. 19b). Notably, Spy1 exerted significant effects on proliferation in a p53-independent manner in the absence of damage; however following triggering of the DDR Spy1 effects were p53 dependent. No significant effects on apoptosis (as shown in FIGS. 19a and 19b, grey bars) occurred at these dose/time regimen.

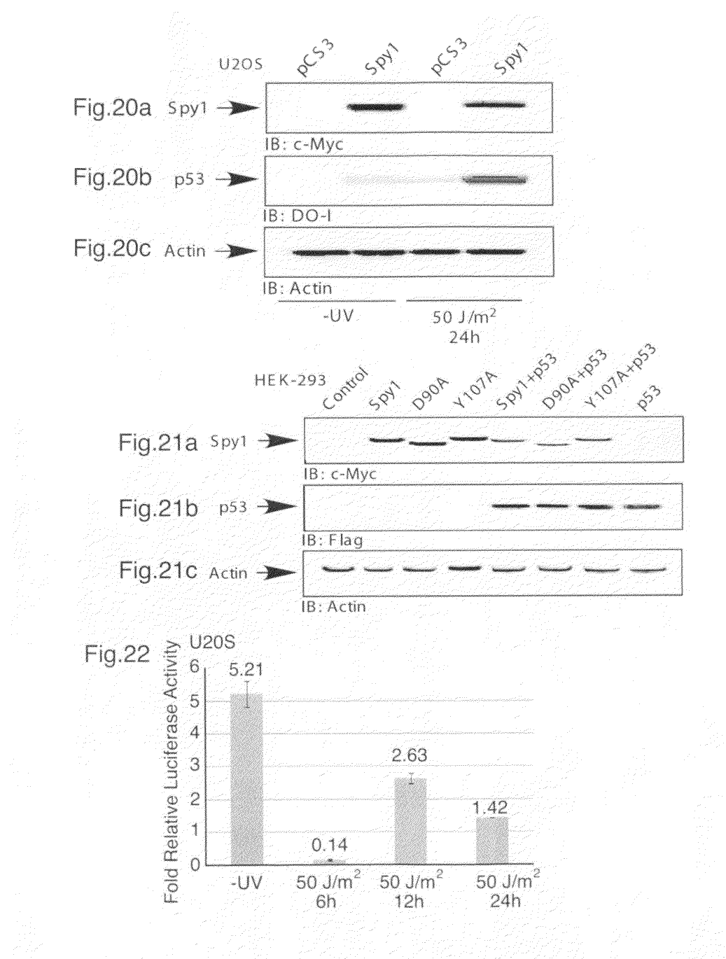

Throughout the experiments Spy1 overexpression markedly increases overall protein levels of p53 (FIGS. 7a to 7c, 8a to 8c, 12a to 12c, and 20a to 20c). The western blots for FIGS. 20a to 20c were obtained using monoclonal c-Myc and DO-1 antibodies. Using binding mutants of Spy1 unable to interact with CDK2 (Spy1D90A; Spy1Y107A) we demonstrate that these effects are not dependent on the direct interaction between Spy1 and CDK2 (as shown in FIGS. 21a to 21c). The western blots for FIGS. 21a to 21c were performed 24 hours after transfection. Hence, Spy1 does not override p53-mediated effects through altering the protein stability of p53.

We then tested the activity of p53 using a luciferase reporter construct (PG13-Luc) containing 13 copies of the p53 consensus binding sequence, and a control reporter plasmid (MG15-Luc). The luciferase reporter assay was performed 12 hours after the cells were either mock treated or treated with UVC radiation (50 J/m²). The luciferase activity is expressed as folds of normalized luciferase activity (normalized to control MG15-Luc) with Spy1 to with pCS3. The relative luciferase activity of pCS3 was assigned the value of 1.0. Each bar represents mean±S.D. (n=3). Interestingly, in the absence of DNA damage we repeatedly observe that Spy1 significantly enhances the transcriptional activity of p53 (as shown in FIG. 22; first lane). However, during damage Spy1 significantly decreases the luciferase activity to less than control (designated as 1), increased activity was then seen for later time points. Hence, Spy1 significantly delays the transcriptional activities of p53 necessary to initiated cellular senescence programs at this dose of UV irradiation.

The methods which are used to obtain the results provided above are provided as follows:

### 1. Cell Culture

Human foreskin fibroblasts (HFF-1) were cultured in Dulbecco's Modified Eagle's Medium (DMEM; D5796; Sigma) supplemented with 15% fetal bovine serum (FBS; F1051; Sigma). Human embryonic kidney cells, HEK-293 (293; CRL-1573; ATCC) and Phoenix cells (ATCC) were maintained in DMEM medium containing 2 mM L-glutamine and 10% FBS (Sigma). The human osteosarcoma cells (U2OS/Saos-2) were cultured in McCoy's 5A 1× (10-050-CV; Cellgro-Mediatech), with 10% FBS. NIH/3T3s were cultured in DMEM supplemented with 10% calf serum (C8056; Sigma). All cells were supplemented with 1% Penicillin and Streptomycin (P/S), and were maintained in an atmosphere of 5% CO₂ at 37° C.

### 2. Plasmids and Transfection

Creation of Myc-Spy1A-PCS3 vector and flag-Spy1A-pLXSN are carried out using methods known to a person skilled in the art. Mutation constructs of Myc-Spy1A-pCS3 encoding D90A and Y107A were also produced using methods known to a person skilled in the art. Spy1 A-DMA was constructed by introducing a new restriction site for EcoRI and inserting a linker (AATTCTCGAGCTCACAACG) (SEQ ID NO: 1) in original Myc-Spy1A-pCS3 plasmid. Phosphorylation mutant p53, S315A, plasmid was generated by site-directed mutagenesis using Flag-p53-pcDNA3 as the template. PG13-Luc and MG15-Luc plasmids were transiently transfected using polyethylenimine (branched PEI; Sigma). In brief, 5-10 μg plasmid DNA was reconstituted in 50 μl/ml of 150 mM NaCI. In a separate tube, 3-5 μl of 10 mg/ml PEI was diluted in 50 μl/ml of 150 mM NaCl and, after a 5 min. incubation, was combined with the DNA solution. The PEI-DNA mixture was incubated for 30 min. at RT and then was gently added, mixed and incubated at RT for 30 min. to allow PEI/DNA complex formation. After a 30 min incubation, the mixture was added dropwise to the tissue culture plate. Cells were incubated in 5% CO₂ for 8 h, and then returned to normal culture medium.

### 3. Generation of Stable Cell Lines

Virus was generated via transfection into Phoenix packaging cells using methods known to a person skilled in the art. Culture supernatant was collected and sterile filtered at 0.45 μm to remove cell debris. HFF-1 cells were infected with virus:culture media ratio of 1:1, supplemented with 0.025 mg/ml polybrene and incubated for 8 hrs. Cells recovered for 24 h in their relevant culture media prior to addition of 400 μg/ml G418.

### 4. Cell Growth/Viability Assays

The number of mean population doublings until senescence was determined via trypan blue exclusion cell counting at each passage as well as cell morphology using light microscopy. Entry into senescence was assessed by in situ senescence-associated β-galactosidase (SA-β-gal) staining at each passage using Senescence Cells Histochemical Staining Kit (CS0030; Sigma). Trypan blue analysis for alive and dead cells was measured after treatment with varying amounts of ultraviolet radiation using a GS Gene Linker (Bio Rad).

### 5. Compounds and Antibodies

The following antibodies were used at the specified dilutions: Spy1A (NB 100-2521; Novus): 1:500, Myc (9E10 and C19; Santa Cruz): 1:1000, Actin (MAB1501R; Chemicon): 1:1000, IgG (se-66186; Santa Cruz): 1:1000, p2I (sc-397; Santa Cruz): 1:100, p53 (D0-1 and 9282; Santa Cruz): 1:1000, p53 (FL-393; Santa Cruz): 1:1000, phospho-S315-p53 (A00485, GenScript): 1:1000, FLAG (F1804; Sigma): 1:2000, CDK2 (M2): 1:100, CDK2 (D-12): 1:1000, GAPDH (0411; Santa Cruz): 1:1000. Secondary antibodies used were HRP-conjugated anti-mouse (A9917; Sigma): 1:10000 and anti-rabbit (A0545: Sigma): 1:10000 IgG. Alexa Fluor 488 (A11008; Invitrogen): 1:1000, Alexa Fluor 488 (A11059; Invitrogen): 1:1000, Hoechst (861405; Sigma): 1:1000. The following compounds were used: MG132 (C2211; Sigma), Cycloheximide (C7698; Sigma), UCN-01 (U6508; Sigma), Chk2 Inhibitor II (C3742, Sigma).

### 6. Immunoblotting and Immunoprecipitation

Samples were lysed with a 0.1% NP40 buffer supplemented with Leupeptin (5 μg/ml), Aprotinin (5 μg/ml) and PMSF (100 μg/ml). Samples were analyzed by 10% SDS-PAGE then transferred to a PVDF membrane. Primary antibodies were applied and incubated at dilutions specified above. Secondary antibodies were used at 1:10,000. Proteins were detected via treatment with Perkin-Elmer Enhanced Chemiluminescence reagent and quantitated with FlourChem HD2 software (Alphalnnotech; Perkin Elmer).

### 7. Immunocytochemistry

Cells were fixed in 4% paraformaldehyde for 1 h, followed by permeablization with a 0.2% triton X solution for 3 min. Fixed cells were blocked for 1 h in 5% FBS and then incubated in primary antibody for 1 h. Cells were then washed 3× in PBS and incubated with Alexa Fluor 488-conjugated secondary antibodies for 1 h. Cells were washed 3× with PBS and then mounted onto glass slides using permount reagent (SP15; Fisher Scientific).

### 8. Luciferase Assays

Cells were harvested 24 h post-transfection with luciferase constructs and mixed with Bright-glo reagent (E2620; Promega). Luminescence spectra of the samples were measured using a plate reader (Wallac Victor 1420; PerkinElmer 3TM-1420).

### 9. Pulse-Chase Radiolabeling:

p21 expressing and p21 co-expressing Spy1 cells were incubated in DMEM without methionine and cystein (D0422; Sigma) containing 5% dialyzed FBS (12105C; Sigma) for 1 h and then switched to medium containing S³⁵-Met/Cys for an additional 4 h, followed by chase periods up to 10 hours in normal medium. At the end of each chase period, cells were lysed and run on a 10% SDS-PAGE gel. Radiolabeled proteins were detected by autoradiography to monitor the half life of the protein using a Cyclone Storage Phosphore System (Perkin Elmer). The densitometric analyses of the bands were quantitated with the OptiQuant software.

### 10. Kinase Assays:

Cells were washed with cold 1× PBS, lysed in 0.1% NP40 lysis buffer and centrifuged at 10,000 g for 10 min. 500 ug of protein was incubated overnight at 4° C. in 500 RI of 0.1% NP40 lysis buffer with 10 ug of anti-CDK2 antibody followed by a 2 h incubation with protein G sepharose beads (17-0618-01; GE Healthcare). Immunocomplexes were washed 3× with 1 ml 0.1% NP40 buffer, aspirated to 50 μl and 50 μl of 2× kinase assay buffer [50 mM Tris-HCI (pH 7.4), 20 mM EGTA, 10 mM MgCl₂ 1 mM DTT, 1 mM sodium orthovanadate] containing 5 μCi of [γ-³²P]ATP (PerkinElmer) was added. Upon addition of 2 μg of histone H1 (382150; CALBIOCHEM) the mixtures were incubated at 30° C. for 30 min. Reactions were terminated with 4× sample buffer, boiled for 5 min and subjected to 12.5% SDS-PAGE. Bands were exposed to a tritium-sensitive phosphor-imaging screen were quantified with the OptiQuant software.

### 11. Statistical Analysis:

Student t test was employed using Statistica software. All results are expressed as mean±SD and differences were considered significant at p values of <0.05.

We claim:

1. A method of downregulating a Spy1 protein in a cell, the method comprising ₜhe step of increasing at least one of a p53 protein and a Chk2 protein in the cell, wherein the p53 protein causes the Chk2 protein to cause degradation of the Spy1 protein.

2. The method of claim 1, wherein the Chk2 protein causes the degradation of the Spy1 protein by a modification of the Spy1 protein.

3. The method of claim 2, wherein the modification occurs at amino acids 217 to 222 of the Spy1 protein.

4. The method of claim 1, wherein the Spy1 protein is degraded by a 26S proteosome.

5. The method of claim 4, wherein the Spy1 protein is targeted for the degradation in an N-terminal region by a ubiquitin.

6. The method of claim 1, wherein the p53 protein in the cell is increased to an amount selected to inhibit or reduce cellular replication.

7. The method of claim 6, wherein the amount is selected to treat cancer.

8. The method of claim 7, wherein the cancer is caused by delayed entry of the cell into cellular senescence.

9. Use of a p53 protein in an amount selected to downregulate a Spy1 protein in a cell, wherein the p53 protein causes a Chk2 protein to cause degradation of the Spy1 protein.

10. The use of claim 9, wherein the Chk2 protein causes the degradation of the Spy1 protein by a modification of the Spy1 protein.

11. The use of claim 10, wherein the modification occurs at amino acids 217 to 222 of the Spy1 protein.

12. The use of claim 9, wherein the Spy1 protein is degraded by a 26S proteosome.

13. The use of claim 12, wherein the Spy1 protein is targeted for the degradation in an N-terminal region by a ubiquitin.

14. The use of claim 9, wherein the amount is selected to inhibit or reduce cellular replication.

15. The use of claim 9, wherein the amount is selected to treat cancer.

16. The use of claim 15, wherein the cancer is caused by delayed entry of the cell into cellular senescence.

17. A method of treating or preventing cancer, the method comprising the step of administering a therapeutically effective amount of an agent selected to downregulate a Spy1 protein in a cell.

18. The method of claim 17, wherein the cancer is a cancer caused by delayed entry of the cell into cellular senescence.

19. The method of claim 17, wherein the agent comprises at least one of a p53 protein, a Chk2 protein, and a S26 proteosome.

20. The method of claim 19, wherein the agent comprises the p53 protein, the Chk2 protein and the S26 proteosome, wherein the p53 protein is present in an amount selected to cause the Chk2 protein to cause degradation of the Spy1 protein.

21. Use of an agent for the treatment or prevention of cancer, wherein the agent is selected to downregulate a Spy1 protein in a cell.

22. The use of claim 21, wherein the cancer is caused by delayed entry into cellular senescence.

23. The use of claim 22, wherein the agent comprises at least one of a p53 protein, a Chk2 protein, and a S26 proteosome.

24. The use of claim 23, wherein the agent comprises the p53 protein, the Chk2 protein and the S26 proteosome, and wherein the p53 protein is present in an amount selected to cause the Chk2 protein to cause degradation of the Spy1 protein.

25. A method of diagnosing or monitoring cancer, the method comprising the steps of extracting a cell from a patient, treating the cell with UV radiation, and measuring amounts of a Spy1 protein and a p53 protein, or a ratio thereof.

26. The method of claim 25, wherein the cancer is caused by delayed entry into cellular senescence.

27. The method of claim 25, wherein the UV radiation comprises a dose of 50 J/m² of UVC radiation.

28. The method of any one of claim 25, wherein the amounts and the ratio are measured at different time points.


SEQUENCE LISTING (2 sequences)

SEQ ID NO: 1 - DNA, 19 bp, Artificial Sequence
  A linker in original Myc-Spy1A-pCS3 plasmid
aattctcgagctcacaacg

SEQ ID NO: 2 - protein, 6 aa, Artificial Sequence
  Amino acids 217 to 222 of Spy1
LPRGPS